(19) United States
(12) Patent Application Publication
(10) Pub. No.: US 2026/0250735 A1
(43) Pub. Date: Aug. 27, 2026

(54) Methanogen Detection System and Method of Detecting Methanogen

(71) Applicant: Carollo Engineers, Inc., Costa Mesa, CA (US)

(72) Inventors: Vikas Kumar, Vancouver, CA (US); Hitendra Kumar, Simrol (IN)

(21) Appl. No.: 19/544,773

(22) Filed: Feb. 19, 2026

Related U.S. Application Data
(60) Provisional application No. 63/761,423, filed on Feb. 21, 2025.
(60) Provisional application No. 63/761,454, filed on Feb. 21, 2025.

(51) Int. Cl. C12Q 1/04; C12Q 1/00

(52) U.S. Cl. CPC C12Q 1/04; C12Q 1/008

(57) ABSTRACT

A methanogen detection system that includes a sample chamber configured to receive a capillary flow medium. The capillary flow medium is configured to support a sample thereon. The sample includes methanogens and each of the methanogens includes Cofactor F₄₂₀. A light source is configured to produce a first light, the light source upstream to the sample chamber, wherein the Cofactor F₄₂₀ of each of the methanogens absorbs the first light produced by the light source thereby resulting in excitation and relaxation of the Cofactor F₄₂₀ of each of the methanogens. Excitation and relaxation of the Cofactor F₄₂₀ of each of the methanogens generates a second emission light from the Cofactor F₄₂₀ of each of the methanogens. A first light detector is configured to detect an intensity of the second emission light.

### CROSS REFERENCE TO RELATED APPLICATION

This application claims priority to U.S. Provisional Patent Application No. 63/761,423 filed Feb. 21, 2025, and U.S. Provisional Patent Application No. 63/761,454 filed Feb. 21, 2025, the entire contents of each of which are herein incorporated by reference.

### BACKGROUND

### 1. Technical Field

The present disclosure relates to systems and methods for investigating or analyzing materials by determining their chemical or physical properties or aspects of their composition. In particular, the present disclosure relates to systems and methods for investigating or analyzing microbial communities within, and/or chemical/physical properties of, samples obtained from wastewater and/or biosolids treatment processes.

### 2. Technical Considerations

The management of wastewater treatment (WWT) and biosolids is an essential aspect of modern sanitation infrastructure, ensuring the safe disposal and reuse of wastewater byproducts. Anaerobic digestion (AD) is a popular method for treating wastewater and biosolids, which utilizes microbial communities to decompose organic matter and produce methane as a valuable renewable energy source. The process of AD relies on the activity of microorganisms, particularly methanogens, which are responsible for converting organic substrates into methane. Methanogens are a class of archaea that have a crucial role in the final stage of AD. They convert volatile fatty acids and hydrogen produced during organic matter degradation into methane gas. Since the abundance of methanogens typically falls within the 1-8% range in most wastewater sludge bioreactors, it represents a bottleneck of AD.

Recent advances in molecular methodologies have greatly increased our knowledge of biological processes, but methanogenic archaea have been largely neglected. Quantitative analysis of methanogens can help maximize process performance, uncover upsets before reactor failure, and ultimately lead to higher resource recovery from the AD systems. Despite the critical role of methanogens, there are significant challenges associated with their monitoring and management.

First, the current molecular instruments and quantitative methods, like DNA sequencing and qPCR (Quantitative Polymerase Chain Reaction) based analysis, demand highly skilled personnel, involve laborious processes, and operate at a slow pace, posing challenges for real-time monitoring. Second, the expense and resource requirements associated with traditional detection methods limit their practicality for routine monitoring in wastewater treatment plants (WWTPs). These methods are expensive, require highly trained personnel, and provide delayed results, making real-time process control difficult. Third, inefficient methanogen monitoring due to delayed results can lead to suboptimal bioreactor analysis, resulting in inviable results which can impact the performances and thereby increase the overall operational costs.

Therefore, there remains a critical need for monitoring and understanding the dynamics of methanogenic populations to optimize AD processes, mitigate process upsets, and maximize biogas production.

### SUMMARY

According to some non-limiting embodiments or aspects, provided is a methanogen detection system that may include: a sample chamber configured to receive a capillary flow medium, the capillary flow medium configured to support a sample thereon, the sample including methanogens and each of the methanogens including Cofactor F₄₂₀; a light source configured to produce a first light, the light source upstream to the sample chamber, where the Cofactor F₄₂₀ of each of the methanogens absorbs the first light produced by the light source thereby resulting in excitation and relaxation of the Cofactor F₄₂₀ of each of the methanogens, and where excitation and relaxation of the Cofactor F₄₂₀ of each of the methanogens generates a second emission light from the Cofactor F₄₂₀ of each of the methanogens; an optical filter downstream from the second emission light, where the optical filter may be configured to transmit a band of the second emission light; a first light detector downstream from the second emission light and the optical filter, where the first light detector may be configured to detect an intensity of the second emission light transmitted through the optical filter; and a computing device in communication with the first light detector, where the computing device may be configured to generate a concentration of the methanogens in the sample based on the intensity of the second emission light detected by the first light detector.

According to some non-limiting embodiments or aspects, the methanogen detection system may further include: the capillary flow medium within the sample chamber.

According to some non-limiting embodiments or aspects, the capillary flow medium may include: a sample loading portion; a hydrophobic partition downstream from the sample loading portion; and a control portion downstream from the hydrophobic partition.

According to some non-limiting embodiments or aspects, the methanogen detection system may further include: a second light detector downstream from the optical filter, where the second light detector is configured to detect an intensity of a third light produced by the light source and transmitted through the optical filter from the control potion of the capillary flow medium.

According to some non-limiting embodiments or aspects, the methanogen detection system may further include: a power supply operatively connected to each of the light source, the first and second light detector, and the computing device.

According to some non-limiting embodiments or aspects, the power supply may be a rechargeable battery.

According to some non-limiting embodiments or aspects, the methanogen detection system may further include: a housing enclosing each of the sample chamber, the light source, the optical filter, the first and second light detector, the computing device, and the power supply.

According to some non-limiting embodiments or aspects, the housing may define an aperture extending through the housing, the aperture including a window configured to provide a view of the sample chamber.

According to some non-limiting embodiments or aspects, the housing may define a capillary flow medium slot, the slot configured to receive the capillary flow medium.

According to some non-limiting embodiments or aspects, the sample loading portion may be configured to receive the sample, where the sample may be configured to flow on the sample loading portion toward the hydrophobic partition, and where the hydrophobic partition may be configured to restrain the sample from flowing to the control portion.

According to some non-limiting embodiments or aspects, the first light produced by the light source including a wavelength range of 420±2.5 nm.

According to some non-limiting embodiments or aspects, the optical filter may be configured to transmit a band of the second emission light including a wavelength range of 470±2.5 nm.

According to some non-limiting embodiments or aspects, the light source may include a plurality of light-emitting diodes (LEDs).

According to some non-limiting embodiments or aspects, each of the first and second light detector may include a photodiode.

According to some non-limiting embodiments or aspects, the sample may further include a lysis solution configured to break down a cell wall of each of the methanogens.

According to some non-limiting embodiments or aspects, the methanogen detection system, may further include: a port extending through the housing and operatively connected to each of the light source, the first and second light detector, and the computing device, where the port may be configured to receive and transfer power and receive, transfer, and output data.

According to some non-limiting embodiments or aspects, the capillary flow medium may further include: a user handling portion separate from the sample loading portion.

According to some non-limiting embodiments or aspects, the control portion may be loaded with at least one fluorophore or at least one fluorochrome.

According to some non-limiting embodiments or aspects, the capillary flow medium may be at least one of the following: chromatography paper, a lateral flow device, a lateral flow strip, or any combination thereof.

According to some non-limiting embodiments or aspects, the housing may include: a first chamber enclosing each of the capillary flow medium, the sample chamber, the light source, the optical filter, and the first and second light detector; and a second chamber enclosing each of the computing device and the power supply.

According to some non-limiting embodiments or aspects, provided is a method of detecting methanogen using the methanogen detection system as described above, including: providing a sample onto a capillary flow medium, the sample including one or more methanogens and each of the methanogens including Cofactor F₄₂₀; providing the capillary flow medium into the sample chamber of the methanogen detection system; irradiating the sample with the first light of the methanogen detection system; detecting an intensity of a second emission light emitted from the Cofactor F₄₂₀ of each of the methanogens; and generating a concentration of the methanogens in the sample based on the intensity of the second emission light.

Further non-limiting embodiments or aspects are set forth in the following numbered clauses:
Clause 1: A methanogen detection system, comprising: a sample chamber configured to receive a capillary flow medium, the capillary flow medium configured to support a sample thereon, the sample comprising methanogens and each of the methanogens comprising Cofactor F₄₂₀; a light source configured to produce a first light, the light source upstream to the sample chamber, wherein the Cofactor F₄₂₀ of each of the methanogens absorbs the first light produced by the light source thereby resulting in excitation and relaxation of the Cofactor F₄₂₀ of each of the methanogens, and wherein excitation and relaxation of the Cofactor F₄₂₀ of each of the methanogens generates a second emission light from the Cofactor F₄₂₀ of each of the methanogens; an optical filter downstream from the second emission light, wherein the optical filter is configured to transmit a band of the second emission light; a first light detector downstream from the second emission light and the optical filter, wherein the first light detector is configured to detect an intensity of the second emission light transmitted through the optical filter; and a computing device in communication with the first light detector, wherein the computing device is configured to generate a concentration of the methanogens in the sample based on the intensity of the second emission light detected by the first light detector.
Clause 2: The methanogen detection system of clause 1, further comprising: the capillary flow medium within the sample chamber.
Clause 3: The methanogen detection system of clause 1 or 2, wherein the capillary flow medium comprises: a sample loading portion; a hydrophobic partition downstream from the sample loading portion; and a control portion downstream from the hydrophobic partition.
Clause 4: The methanogen detection system of any of clauses 1-3, further comprising: a second light detector downstream from the optical filter, wherein the second light detector is configured to detect an intensity of a third light produced by the light source and transmitted through the optical filter from the control potion of the capillary flow medium.
Clause 5: The methanogen detection system of any of clauses 1-4, further comprising: a power supply operatively connected to each of the light source, the first and second light detector, and the computing device.
Clause 6: The methanogen detection system of any of clauses 1-5, wherein the power supply is a rechargeable battery.
Clause 7: The methanogen detection system of any of clauses 1-6, further comprising: a housing enclosing each of the sample chamber, the light source, the optical filter, the first and second light detector, the computing device, and the power supply.
Clause 8: The methanogen detection system of any of clauses 1-7, wherein the housing defines an aperture extending through the housing, the aperture comprising a window configured to provide a view of the sample chamber.
Clause 9: The methanogen detection system of any of clauses 1-8, wherein the housing defines a capillary flow medium slot, the slot configured to receive the capillary flow medium.
Clause 10: The methanogen detection system of any of clauses 1-9, wherein the sample loading portion is configured to receive the sample, wherein the sample is configured to flow on the sample loading portion toward the hydrophobic partition, and wherein the hydrophobic partition is configured to restrain the sample from flowing to the control portion.
Clause 11: The methanogen detection system of any of clauses 1-10, wherein the first light produced by the light source comprises a wavelength range of 420±2.5 nm.
Clause 12: The methanogen detection system of any of clauses 1-11, wherein the optical filter is configured to transmit a band of the second emission light comprising a wavelength range of 470±2.5 nm.
Clause 13: The methanogen detection system of any of clauses 1-12, wherein the light source comprises a plurality of light-emitting diodes (LEDs).
Clause 14: The methanogen detection system of any of clauses 1-13, wherein each of the first and second light detector comprises a photodiode.
Clause 15: The methanogen detection system of any of clauses 1-14, wherein the sample further comprises a lysis solution configured to break down a cell wall of each of the methanogens.
Clause 16: The methanogen detection system of any of clauses 1-15, further comprising: a port extending through the housing and operatively connected to each of the light source, the first and second light detector, and the computing device, wherein the port is configured to receive and transfer power and receive, transfer, and output data.
Clause 17: The methanogen detection system of any of clauses 1-16, wherein the capillary flow medium further comprises: a user handling portion separate from the sample loading portion.
Clause 18: The methanogen detection system of any of clauses 1-17, wherein the control portion is loaded with at least one fluorophore or at least one fluorochrome.
Clause 19: The methanogen detection system of any of clauses 1-18, wherein the capillary flow medium is at least one of the following: chromatography paper, a lateral flow device, a lateral flow strip, or any combination thereof.
Clause 20: The methanogen detection system of any of clauses 1-19, wherein the housing comprises: a first chamber enclosing each of the capillary flow medium, the sample chamber, the light source, the optical filter, and the first and second light detector; and a second chamber enclosing each of the computing device and the power supply.
Clause 21: A method of detecting methanogen using the methanogen detection system of any of clauses 1-20, comprising: providing a sample onto a capillary flow medium, the sample comprising one or more methanogens and each of the methanogens comprising Cofactor F₄₂₀; providing the capillary flow medium into the sample chamber of the methanogen detection system; irradiating the sample with the first light of the methanogen detection system; detecting an intensity of a second emission light emitted from the Cofactor F₄₂₀ of each of the methanogens; and generating a concentration of the methanogens in the sample based on the intensity of the second emission light.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosed subject matter.

### BRIEF DESCRIPTION OF THE DRAWINGS

Additional advantages and details are explained in greater detail below with reference to the non-limiting, exemplary embodiments that are illustrated in the accompanying schematic figures, in which:

FIG. 1 is a perspective, exploded view of a methanogen detection system, according to some non-limiting embodiments or aspects of the present disclosure;

FIG. 2 is a perspective view of the methanogen detection system of FIG. 1 before a capillary flow medium is received in a sample chamber of the system, according to some non-limiting embodiments or aspects of the present disclosure;

FIG. 3 is a perspective view of the methanogen detection system of FIGS. 1 and 2, according to some non-limiting embodiments or aspects of the present disclosure;

FIG. 4 is a graphical representation showing a relationship between cell dry mass of methanogens and relative fluorescence units (RFU), which can be determined by measuring light emitted from the Cofactor F₄₂₀ of each of the methanogens; and

FIG. 5 is a schematic diagram of example components of the methanogen detection system of FIGS. 1-3.

Corresponding reference characters indicate corresponding features throughout the several views of the drawings. The representations set out herein illustrate exemplary aspects of the disclosure, and such representations are not to be construed as limiting the scope of the disclosure in any manner.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. In addition, reference to an action being “based on” a condition may refer to the action being “in response to” the condition. For example, the phrases “based on” and “in response to” may, in some non-limiting embodiments or aspects, refer to a condition for automatically triggering an action (e.g., a specific operation of an electronic device, such as a computing device, a processor, and/or the like).

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

As used herein, the phrase “in communication” may refer to a relationship capable of reception, receipt, transmission, transfer, provision, and/or the like of data (e.g., information, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit processes information received from the first unit and communicates the processed information to the second unit.

As used herein, the term “system” may refer to one or more computing devices or combinations of computing devices (e.g., processors, servers, client devices, software applications, components of such, and/or the like). Reference to “a device,” “a server,” “a processor,” and/or the like, as used herein, may refer to a previously-recited device, server, or processor that is recited as performing a previous step or function, a different device, server, or processor, and/or a combination of devices, servers, and/or processors. For example, as used in the specification and the claims, a first device, a first server, or a first processor that is recited as performing a first step or a first function may refer to the same or different device, server, or processor recited as performing a second step or a second function.

While this disclosure is made as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

In some non-limiting embodiments or aspects, the present disclosure proposes microfluidic systems as transformative platforms for transferring complex laboratory analytical procedures onto a single detection system, which offers distinct advantages such as minimal reagent consumption, enhanced efficiency, and rapid detection. This microfluidic approach presents a straightforward and cost-effective avenue for investigating crucial performance parameters in AD systems. These methods enable the visualization of methanogenic archaea through their intrinsic fluorescent Cofactor F₄₂₀. The present disclosure explores the auto-fluorescence of the methanogens'Cofactor F₄₂₀ to quantify and enumerate their growth profiles, and the present disclosure provides the capability to study the health of AD systems. Determining the relative abundance of methanogens in engineered anaerobic systems provides crucial onsite results for discovering disturbances before failure or for improvising the overall process.

In some non-limiting embodiments or aspects, analyzing the auto-fluorescent properties of Cofactor F₄₂₀ offers a foundation for the effectiveness of a methanogen detection system 10 and method of detecting methanogen. The methanogen detection system 10 and method of detecting methanogen of the present disclosure hold immense potential as tools for scientists and water utilities working to develop more efficient and sustainable treatment processes. The methanogen detection system 10 and method of detecting methanogen of the present disclosure represent a critical need in the field of wastewater and biosolids treatment for, at least, the following reasons.

Efficiency and Cost-Effectiveness: In some non-limiting embodiments or aspects, the methanogen detection system 10 and method of detecting methanogen of the present disclosure offer opportunities for rapid, cost-effective, and user-friendly solutions for monitoring methanogen populations in anaerobic digesters, allowing WWTPs to optimize their operations and reduce maintenance costs.

Real-Time Monitoring: In some non-limiting embodiments or aspects, real-time monitoring of methanogens is essential for promptly identifying shifts in microbial communities or process upsets, allowing for immediate corrective actions to be taken to avoid digester failures.

Regulatory Compliance: In some non-limiting embodiments or aspects, numerous regulatory bodies mandate that WWT facilities adhere to specific performance standards, including methane production efficiency. The methanogen detection system 10 and method of detecting methanogen of the present disclosure facilitate compliance with these regulations, ensuring that facilities meet their environmental obligations.

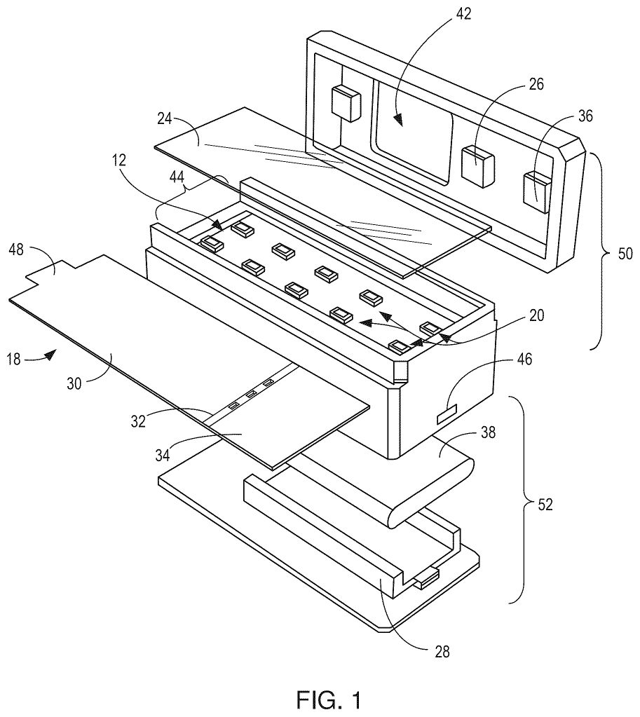

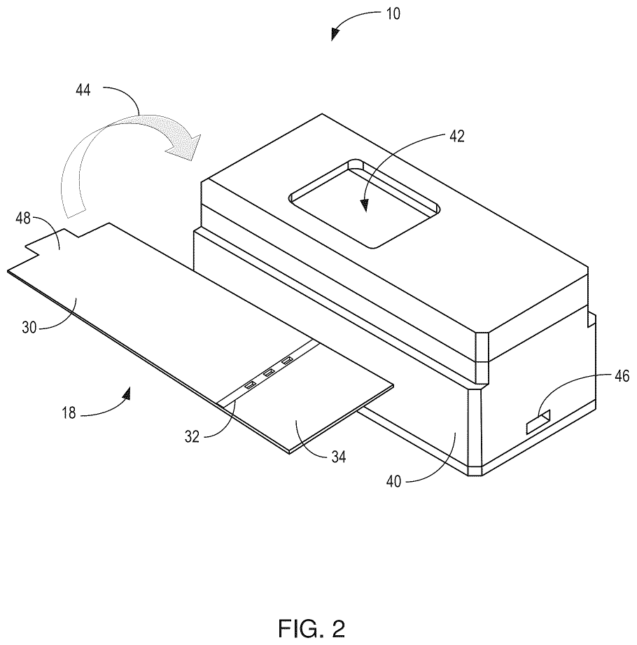

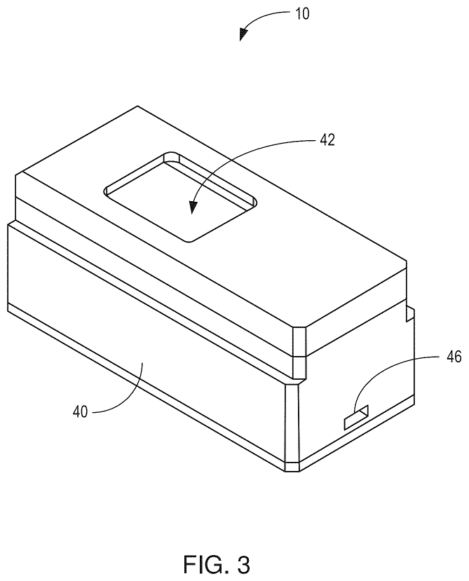

Referring to FIG. 1, in some non-limiting embodiments or aspects, there is shown a perspective, exploded view of a methanogen detection system 10, according to some non-limiting embodiments or aspects of the present disclosure. Referring to FIG. 2, there is shown a perspective view of the methanogen detection system 10 of FIG. 1 before a capillary flow medium 18 is received in a sample chamber 12 of the system 10, according to some non-limiting embodiments or aspects of the present disclosure. Referring to FIG. 3, there is shown a perspective view of the methanogen detection system 10 of FIGS. 1 and 2, according to some non-limiting embodiments or aspects of the present disclosure.

As shown in FIGS. 1-3, in some non-limiting embodiments or aspects, the methanogen detection system 10 includes a sample chamber 12 configured to receive a capillary flow medium 18. The capillary flow medium 18 is configured to support a sample thereon. The sample includes methanogens and each of the methanogens includes Cofactor F₄₂₀. The methanogen detection system 10 includes a light source 20 configured to produce a first light. The light source 20 is upstream to the sample chamber 12. The Cofactor F₄₂₀ of each of the methanogens absorbs the first light produced by the light source 20 thereby resulting in excitation and relaxation of the Cofactor F₄₂₀ of each of the methanogens. Excitation and relaxation of the Cofactor F₄₂₀ of each of the methanogens generates a second emission light from the Cofactor F₄₂₀ of each of the methanogens. The methanogen detection system 10 includes an optical filter 24 downstream from the second emission light. The optical filter 24 is configured to transmit a band of the second emission light. The methanogen detection system 10 includes a first light detector 26 downstream from the second emission light and the optical filter 24. The first light detector 26 is configured to detect an intensity of the second emission light transmitted through the optical filter 24. The methanogen detection system 10 includes a computing device 28 in communication with the first light detector 26. The computing device 28 is configured to generate a concentration of the methanogens in the sample based on the intensity of the second emission light detected by the first light detector 26.

In some non-limiting embodiments or aspects, the methanogen detection system 10 can include a capillary flow medium 18 within the sample chamber 12. In some non-limiting embodiments or aspects, the capillary flow medium 18 can be chromatography paper. In some non-limiting embodiments or aspects, the capillary flow medium 18 can be a lateral flow device or a lateral flow strip. If the capillary flow medium 18 is a lateral flow device or a lateral flow strip, at least a portion of the lateral flow device or strip can be chromatography paper. The capillary flow medium 18 can be configured to support a sample thereon, and the capillary flow medium 18 can allow capillary flow of the sample thereon/therethrough. The sample can include methanogens and each of the methanogens can include Cofactor F₄₂₀. The sample can be a liquid solution. If the sample is a liquid solution, the sample can include a non-polar organic solvent and a polar solvent, such as water. The sample can also include a lysis solution configured to break down a cell wall of each of the methanogens. The lysis solution may or may not be the non-polar organic solvent. In some non-limiting embodiments or aspects, the lysis solution is chloroform and/or acetone. The capillary flow medium 18 not only allows capillary flow of the sample thereon/therethrough, but can also filter cell debris of the methanogens as a result of the breakdown of the cell wall of each of the methanogens by the lysis solution.

In some non-limiting embodiments or aspects, the capillary flow medium 18 includes a sample loading portion 30, a hydrophobic partition 32 downstream from the sample loading portion 30, and a control portion 34 downstream from the hydrophobic partition 32. The sample loading portion 30 can allow capillary flow of the sample thereon/therethrough. The sample loading portion 30 is configured to receive the sample. The sample is configured to flow on the sample loading portion 30 toward the hydrophobic partition 32. The hydrophobic partition 32 is configured to restrain the sample from flowing to the control portion 34. The hydrophobic partition can include a hydrophobe or a hydrophobic material or substance that repels water. In some non-limiting embodiments or aspects, the control portion 34 may be loaded with at least one fluorophore or at least one fluorochrome. That is, a fluorophore or fluorochrome can be deposited on/within the control portion 34 of the capillary flow medium 18. In some non-limiting embodiments or aspects, nothing is loaded on the control portion 34 of the capillary flow medium 18.

In some non-limiting embodiments or aspects, as will be discussed below, the methanogen detection system 10 can include a second light detector 36 downstream from the optical filter 24, and the second light detector 36 can be configured to detect an intensity of a third light produced by the light source 20 that is transmitted through the optical filter 24 from the control potion 34 of the capillary flow medium 18. That is, a third light, that is separate from the first light produced by the light source 20 and separate from the second emission light generated from the Cofactor F₄₂₀ of each of the methanogens, travels from control portion 34 of the capillary flow medium 18, through the optical filter 24, and to the second light detector 36. In some non-limiting embodiments or aspects, a known concentration, volume, or mass of the fluorophore or fluorochrome can be deposited on/within the control portion 34 of the capillary flow medium 18. The fluorophore or fluorochrome deposited on/within the control portion 34 of the capillary flow medium 18 can allow a known and constant intensity and/or wavelength of the third light to be transmitted through the optical filter 24 from the control portion 34 of the capillary flow medium 18. In some non-limiting embodiments or aspects, nothing is loaded on the control portion 34 of the capillary flow medium 18 such that the blank control portion 34 produces a known and constant intensity and/or wavelength of the third light that is transmitted through the optical filter 24 from the control portion 34 of the capillary flow medium 18. In each of these non-limiting embodiments or aspects, the third light detected by the second light detector 36 provides a constant reading of the control portion 34 of the capillary flow medium 18 that is used to obtain a background signal of the capillary flow medium 18 for normalization of the intensity of the second emission light transmitted through the optical filter 24 from the Cofactor F₄₂₀ of each of the methanogens.

In some non-limiting embodiments or aspects, as shown in FIGS. 1 and 2, the capillary flow medium 18 can include a user handling portion 48 separate from the sample loading portion 30. The user handling portion 48 of the capillary flow medium 18 is configured to provide a surface area for the user to handle the capillary flow medium 18 without contaminating the sample loading portion 30. In some non-limiting embodiments or aspects, as shown in FIGS. 1 and 2, the user handling portion 48 can be adjacent to the sample loading portion 30. In some non-limiting embodiments or aspects, the user handling portion 48 can be adjacent to the control portion 34.

In some non-limiting embodiments or aspects, the sample can include a non-polar organic solvent and a polar solvent, such as water. The sample can flow on/through the capillary flow medium 18 which results in the sample separating into its constituent elements. If the capillary flow medium 18 includes chromatography paper, the capillary flow medium 18 can be made of (at least in part) cellulose, and the cellulose of the chromatography paper attracts the polar solvent, such as water, which results in less mobility of the polar solvent. Therefore, the polar solvent is considered to be the stationary phase and the capillary flow medium 18 is considered to support the stationary phase. The components of the sample that are less polar, including the non-polar organic solvent, are less attracted to the cellulose of the capillary flow medium 18. This results in greater mobility of the non-polar components of the sample and the non-polar organic solvent. Thus, the capillary flow medium 18 allows the sample to be separated into its constituent elements based on polarities of the constituent elements.

In some non-limiting embodiments or aspects, the cell debris of the methanogens, resulting from the breakdown of the cell wall of each of the methanogens by the lysis solution, are retained and immobile on/through the capillary flow medium 18. That is, the capillary flow medium 18 can filter the cell debris of the methanogens. In some non-limiting embodiments or aspects, the Cofactor F₄₂₀ of each of the methanogens is mobile on/through the capillary flow medium 18 and travels toward a distal portion of the capillary flow medium 18, such as a distal portion of the sample loading portion 30. The distal portion of the sample loading portion 30 can be near and/or adjacent to the hydrophobic partition 32.

In some non-limiting embodiments or aspects, the methanogen detection system 10 can include the light source 20 configured to produce the first light. The light source 20 can be upstream to the sample chamber 12 and the capillary flow medium 18 when the capillary flow medium 18 is present within the sample chamber 12. In some non-limiting embodiments or aspects, the light source 20 can be a light-emitting diode (LED). In some non-limiting embodiments or aspects, the light source 20 can include a plurality of LEDs. For example, as shown in FIG. 1, the light source 20 can be an LED array. The first light produced by the light source 20 can have, at least, a wavelength range of 420±2.5 nm. In some non-limiting embodiments or aspects, the first light produced by the light source 20 can have various wavelengths, in which one of the wavelengths is approximately 420 nm.

In some non-limiting embodiments or aspects, the Cofactor F₄₂₀ of each of the methanogens absorbs the first light produced by the light source 20 thereby resulting in excitation and relaxation of the Cofactor F₄₂₀ of each of the methanogens. In particular, the Cofactor F₄₂₀ of each of the methanogens absorbs the wavelength of approximately 420 nm of the first light produced by the light source 20 thereby resulting in excitation and relaxation of the Cofactor F₄₂₀ of each of the methanogens. Excitation and relaxation of the Cofactor F₄₂₀ of each of the methanogens generates a second emission light from the Cofactor F₄₂₀ of each of the methanogens. The second emission light generated by excitation and relaxation of the Cofactor F₄₂₀ can have a wavelength range of 470±2.5 nm. Furthermore, the second emission light generated by excitation and relaxation of the Cofactor F₄₂₀ can have various wavelengths, in which one of the wavelengths is approximately 470 nm.

In some non-limiting embodiments or aspects, the above-described process of absorbance of the first light produced by the light source 20, excitation and relaxation, and emittance of the second emission light generated by the excitation and relaxation of the Cofactor F₄₂₀ generally illustrates the process of fluorescence of the Cofactor F₄₂₀ of each of the methanogens. Fluorescence of Cofactor F₄₂₀ occurs through the following specific process. The Cofactor F₄₂₀ molecules of each of the methanogens absorb the first light produced by the light source 20 thereby resulting in excitation of electrons of the Cofactor F₄₂₀ molecules of each of the methanogens. In particular, the Cofactor F₄₂₀ molecules of each of the methanogens absorb the wavelength of approximately 420 nm of the first light produced by the light source 20 thereby resulting in excitation of electrons of the Cofactor F₄₂₀ molecules of each of the methanogens. Excitation of the electrons of the Cofactor F₄₂₀ molecules of each of the methanogens results in the electrons transitioning to a higher energy state. When the electrons of the Cofactor F₄₂₀ molecules relax or return to a lower energy state, the electrons of the Cofactor F₄₂₀ molecules emit photons at a longer wavelength than the wavelength of approximately 420 nm of the first light. The emitted photons make up or constitute the second emission light from the Cofactor F₄₂₀ of each of the methanogens, which is observable as fluorescence. The second emission light generated by the emitted photons from the electrons of the Cofactor F₄₂₀ molecules relaxing or returning to a lower energy state can have a wavelength range of 470±2.5 nm. Furthermore, the second emission light generated by excitation and relaxation of the Cofactor F₄₂₀ can have various wavelengths, in which one of the wavelengths is approximately 470 nm.

In some non-limiting embodiments or aspects, the methanogen detection system 10 can include an optical filter 24 downstream from the second emission light. The optical filter 24 can be configured to transmit a band of the second emission light. The band of the second emission light transmitted by the optical filter 24 can have a wavelength range of 470±2.5 nm. In some non-limiting embodiments or aspects, the optical filter 24 is a band-pass optical filter.

In some non-limiting embodiments or aspects, the methanogen detection system 10 can include a first light detector 26 downstream from the second emission light and the optical filter 24. The first light detector 26 can be configured to detect an intensity of the second emission light transmitted through the optical filter 24. in some non-limiting embodiments or aspects, as shown in FIG. 1, the first light detector 26 can be a photodiode. In some non-limiting embodiments or aspects, the first light detector 26 can be a spectrophotometer or camera. If the first light detector 26 is a photodiode, the photodiode can be a photodiode sensor. Using readily available equipment, such as a photodiode for the first light detector 26, makes the methanogen detection system 10 accessible and cost-effective.

In some non-limiting embodiments or aspects, the methanogen detection system 10 can include a computing device 28. The computing device 28 can be configured to generate a concentration of the methanogens in the sample based on the intensity of the second emission light detected by first the light detector 26. The computing device 28 can be one or more electronic device(s) that is/are configured to process data. Each computing device 28 can include a processor, microprocessor/microcontroller, a user interface, an input device, a display, a memory, a network interface, etc. That is, each computing device 28 can include any components necessary to receive, store, process, and/or output data. in some non-limiting embodiments or aspects, each of the first 26 and second light detector 36 is in communication with the computing device 28. In some non-limiting embodiments or aspects, the computing device 28 is operatively connected to and in communication with at least one of the following: the light source 20, the first 26 and second light detector 36, a power supply 38 (as will be discussed below), or any combination thereof. The computing device 28 can be configured to control the methanogen detection system 10, for example, by controlling system operation, detector recording, and/or data transmission (e.g., wired or wireless). Specifically, the computing device 28 can be programmed to record intensity readings of the second emission light detected by the first light detector 26 on a trigger or automatically at different time intervals.

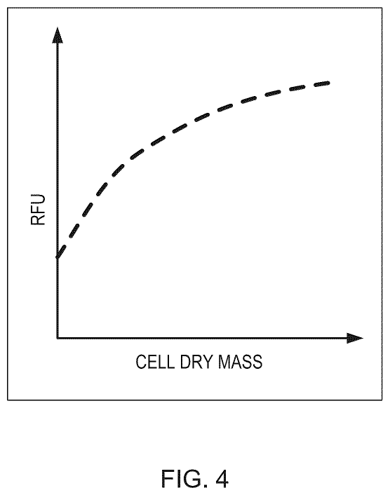

In some non-limiting embodiments or aspects, if the methanogen detection system 10 includes a computing device 28 in communication with the first 26 and second light detector 36, the second emission light transmitted through the optical filter 24 can be recorded and stored in a memory of the computing device 28. The intensity of the second emission light transmitted through the optical filter 24, as the fluorescence signal, can be used to determine the concentration of the methanogens in the sample. As shown in FIG. 4, a calibration curve can be established using known concentrations of methanogens (based on cell dry mass of the methanogens) in various samples and measuring the intensity of the light transmitted through the optical filter 24 (as relative fluorescence units (RFU)) for each of the known concentrations of the methanogens in the various samples. In some non-limiting embodiments or aspects, the calibration curve can be stored in the memory or storage of the computing device 28. In some non-limiting embodiments or aspects, the calibration curve can be incorporated into a computer program product including program instructions that can be executed by a processor of the computing device 28. The computer program product can be a mobile app or web app and can utilize an R software package. As identified above, the third light detected by the second light detector 36 provides a constant reading of the control portion 34 of the capillary flow medium 18 that is used to obtain a background signal of the capillary flow medium 18 for normalization of the intensity of the second emission light transmitted through the optical filter 24 from the Cofactor F₄₂₀ of each of the methanogens. For a sample having an unknown concentration of methanogens, the computing device 28 can apply the calibration curve to the second emission light transmitted through the optical filter 24, and recorded and stored in a memory of the computing device 28, and can determine the concentration of the methanogens in the sample.

In some non-limiting embodiments or aspects, the intensity of the second emission light transmitted through the optical filter 24 can be used to determine the concentration of the methanogens in the sample. In other words, the unknown methanogen concentration in the sample will be calibrated with the known methanogen concentrations based on corresponding emission signals. A computer software or smartphone application can be used to transcribe the image intensity to the corresponding numerical values for concentration. Unknown concentrations within samples can be examined using the calibration curve to determine the relative methanogen concentrations in the samples.

In some non-limiting embodiments or aspects, the sample loading portion 30 of the capillary flow medium 18 can be configured to receive the sample. For example, a user of the methanogen detection system 10 can drop a sample on the sample loading portion 30. The sample then proceeds to flow on/through the sample loading portion 30. Flow of the sample on/through the sample loading portion 30 can be aided at least in part by capillary action. As discussed above, the sample is configured to flow on the sample loading portion 30 toward the hydrophobic partition 32. The hydrophobic partition 32 is configured to restrain the sample from flowing to the control portion 34.

In some non-limiting embodiments or aspects, the computing device 28 can be any computing device that is capable of controlling and/or communicating with any of the light source 20, the first 26 and second light detector 36, and the power supply 38; receiving data from any of the light source 20, the first 26 and second light detector 36, and the power supply 38; storing data, such as data received from any of the light source 20, the first 26 and second light detector 36, and the power supply 38; processing data, such as data received from any of the light source 20, the first 26 and second light detector 36, and the power supply 38; and/or outputting data. Specifically, the computing device 28 can be any computing device that is capable of operating any of the light source 20, the first 26 and second light detector 36, and the power supply 38; receiving power from the power supply 38; communicating with any of the light source 20, the first 26 and second light detector 36, and the power supply 38; receiving the intensity of the second emission light transmitted through the optical filter 24 from the first light detector 26; receiving the intensity of the third light transmitted through the optical filter 24 from the second light detector 36; storing the intensity of the second emission light transmitted through the optical filter 24 and received from the first light detector 26; storing the intensity of the third light transmitted through the optical filter 24 and received from the second light detector 36; processing the intensity of the second emission light transmitted through the optical filter 24 and received from the first light detector 26; processing the intensity of the third light transmitted through the optical filter 24 and received from the second light detector 36; and outputting a concentration of the methanogens in the sample using the calibration curve. Using readily available equipment, makes the methanogen detection system 10 and method of detecting methanogen accessible and cost-effective.

In some non-limiting embodiments or aspects, as described previously, the sample can also include a lysis solution configured to break down a cell wall of each of the methanogens. In some non-limiting embodiments or aspects, the lysis solution is chloroform and/or acetone. The sample flows through the sample loading portion 30 in sufficient time to allow the lysis solution to break down the cell wall of each of the methanogens. Using a lysis solution to break down the cell wall of each of the methanogens allows intracellular Cofactor F₄₂₀ of each of the methanogens of the sample to release to the mobile solution and move on/through the capillary flow medium 18 and travel to a distal portion of the capillary flow medium 18, such as a distal portion of the sample loading portion 30. The distal portion of the sample loading portion 30 can be near and/or adjacent to the hydrophobic partition 32. As identified above, the auto-fluorescence property of Cofactor F₄₂₀ generates emission of photons when incident light having a wavelength of 420 nm is absorbed by the Cofactor F₄₂₀ molecules.

In some non-limiting embodiments or aspects, the methanogen detection system 10 can include a power supply 38 operatively connected to each of the light source 20, the first 26 and second light detector 36, and the computing device 28. The power supply 38 can be a rechargeable battery. The power supply 38 can be configured to supply power to each of the light source 20, the first 26 and second light detector 36, and the computing device 28.

In some non-limiting embodiments or aspects, the methanogen detection system 10 can include a housing 40 enclosing each of the sample chamber 12, the light source 20, the optical filter 24, the first 26 and second light detector 36, the computing device 28, and the power supply 38. As shown in FIGS. 1-3, the housing 40 can define an aperture 42 extending through the housing 40. The aperture 42 can include a window configured to provide a view of the sample chamber 12. Furthermore, the housing 40 can define a capillary flow medium slot 44. The slot 44 can be configured to receive the capillary flow medium 18. The housing 40 can include a first chamber 50 enclosing each of the capillary flow medium 18, the sample chamber 12, the light source 20, the optical filter 24, and the first 26 and second light detector 36. The housing 40 can also include a separate second chamber 52 enclosing each of the computing device 28 and the power supply 38.

In some non-limiting embodiments or aspects, the methanogen detection system 10 can include a port 46 extending through the housing 40 and operatively connected to and in communication with each of the light source 20, the first 26 and second light detector 36, and the computing device 28. The port 46 can be configured to receive and transfer power and receive, transfer, and output data. In some non-limiting embodiments or aspects, the port 46 is a Universal Serial Bus (USB) port. By having the port 46, a user can control/program, receive data from, and transmit data to the components of the methanogen detection system 10, such as the computing device 28.

Referring to FIG. 4, in some non-limiting embodiments or aspects, there is shown a graphical representation showing a relationship between cell dry mass of methanogens and relative fluorescence units (RFU), which can be determined by measuring the second emission light emitted from the Cofactor F₄₂₀ of each of the methanogens. In some non-limiting embodiments or aspects, a standard curve can be created by correlating the Cofactor F₄₂₀ concentration with relative fluorescent units (RFU) values. This standard curve can be incorporated into a computer program product including program instructions that can be executed by the computing device 28. As discussed above, the computing device 28 can be configured to generate a concentration of the methanogens in the sample based on the intensity of the second emission light detected by the first light detector 26. The computer program product can be a mobile app or web app and can utilize an R software package. In some non-limiting embodiments or aspects, the concentration of the Cofactor F₄₂₀ relative to the methanogen concentration in the sample can be determined using reference values.

In some non-limiting embodiments or aspects, the present disclosure is also directed to a method of detecting methanogen. The method of detecting methanogen can include irradiating a sample comprising methanogens and each of the methanogens comprising Cofactor F₄₂₀ with a first light, detecting an intensity of a second emission light emitted from the Cofactor F₄₂₀ of each of the methanogens, and generating a concentration of the methanogens in the sample based on the intensity of the second emission light.

In some non-limiting embodiments or aspects, the method of detecting methanogen can include any of the following elements: adding a lysis solution to the sample to break down a cell wall of each of the methanogens; contacting the sample to a capillary flow medium; filtering the second emission light through an optical filter by transmitting a band of the second emission light having a wavelength range of 470±2.5 nm; and/or detecting an intensity of a third light emitted from the capillary flow medium.

In some non-limiting embodiments or aspects, the methanogen detection system 10 and method of detecting methanogen each provide the following benefits. Each of the methanogen detection system 10 and method provides: guidance to utilities by offering utilities comprehensive insights into biosolid treatment, regulatory perspectives, and advanced removal technologies; facilitates informed decision-making by keeping utilities well-informed with the latest developments in AD and methanogen detection; generates proactive engagement in methanogen detection and biosolid treatment initiatives; offers solutions in methanogen detection, aligning with industry demands and trends; develops and standardizes tools and databases to support engineers as regulatory frameworks evolve; ensures readiness in navigating regulatory changes and provides users with valuable insights; extends support to users in anaerobic bioreactor and biosolid applied research; and/or actively allows for collaboration with utilities to tailor research efforts, fostering innovation and advancing biosolid treatment practices.

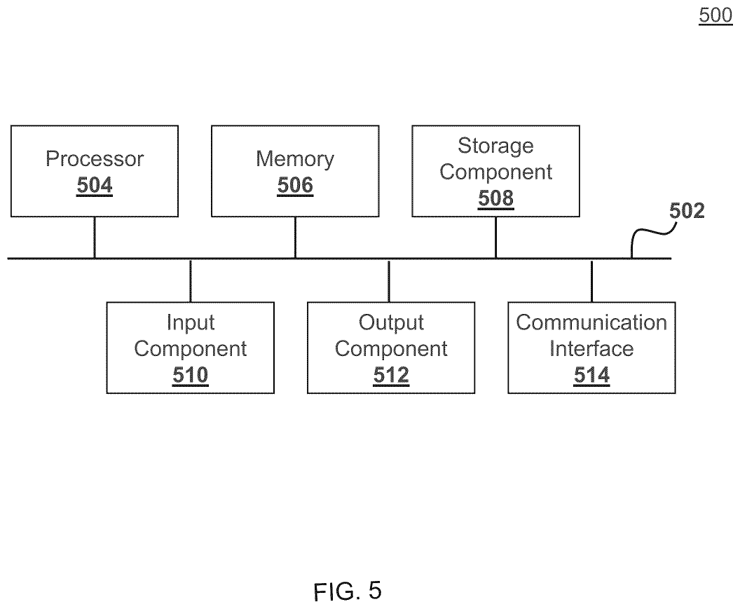

Referring now to FIG. 5, shown is a diagram of example components of device 500, according to non-limiting embodiments or aspects. Device 500 may correspond to at least one of the methanogen detection system 10, the first light detector 26, the computing device 28, the second light detector 36, and/or the port 46 in FIGS. 1-3. In some non-limiting embodiments or aspects, such systems or devices in FIGS. 1-3 may include at least one device 500 and/or at least one component of device 500. The number and arrangement of components shown in FIG. 5 are provided as an example. In some non-limiting embodiments or aspects, device 500 may include additional components, fewer components, different components, or differently arranged components than those shown in FIG. 5. Additionally or alternatively, a set of components (e.g., one or more components) of device 500 may perform one or more functions described as being performed by another set of components of device 500.

As shown in FIG. 5, device 500 may include bus 502, processor 504, memory 506, storage component 508, input component 510, output component 512, and communication interface 514. Bus 502 may include a component that permits communication among the components of device 500. In some non-limiting embodiments or aspects, processor 504 may be implemented in hardware, firmware, or a combination of hardware and software. For example, processor 504 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 506 may include random access memory (RAM), read only memory (ROM), and/or another type of dynamic or static storage device (e.g., flash memory, magnetic memory, optical memory, etc.) that stores information and/or instructions for use by processor 504.

With continued reference to FIG. 5, storage component 508 may store information and/or software related to the operation and use of device 500. For example, storage component 508 may include a hard disk (e.g., a magnetic disk, an optical disk, a magneto-optic disk, a solid-state disk, etc.) and/or another type of computer-readable medium. Input component 510 may include a component that permits device 500 to receive information, such as via user input (e.g., a touch screen display, a keyboard, a keypad, a mouse, a button, a switch, a microphone, etc.). Additionally or alternatively, input component 510 may include a sensor for sensing information (e.g., a global positioning system (GPS) component, an accelerometer, a gyroscope, an actuator, etc.). Output component 512 may include a component that provides output information from device 500 (e.g., a display, a speaker, one or more light-emitting diodes (LEDs), etc.). Communication interface 514 may include a transceiver-like component (e.g., a transceiver, a separate receiver and transmitter, etc.) that enables device 500 to communicate with other devices, such as via a wired connection, a wireless connection, or a combination of wired and wireless connections. Communication interface 514 may permit device 500 to receive information from another device and/or provide information to another device. For example, communication interface 514 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 500 may perform one or more processes described herein. Device 500 may perform these processes based on processor 504 executing software instructions stored by a computer-readable medium, such as memory 506 and/or storage component 508. A computer-readable medium may include any non-transitory memory device. A memory device may include memory space located inside of a single physical storage device or memory space spread across multiple physical storage devices. Software instructions may be read into memory 506 and/or storage component 508 from another computer-readable medium or from another device via communication interface 514. When executed, software instructions stored in memory 506 and/or storage component 508 may cause processor 504 to perform one or more processes described herein. Additionally or alternatively, hardwired circuitry may be used in place of or in combination with software instructions to perform one or more processes described herein. Thus, embodiments described herein are not limited to any specific combination of hardware circuitry and software. The term “configured to,” as used herein, may refer to an arrangement of software, device(s), and/or hardware for performing and/or enabling one or more functions (e.g., actions, processes, steps of a process, and/or the like). For example, “a processor configured to” may refer to a processor that executes software instructions (e.g., program code) that cause the processor to perform one or more functions.

While exemplary designs have been described above in the detailed description, those of ordinary skill in the art will understand that the exemplary designs of the present disclosure can be further modified within the spirit and scope of this disclosure. Therefore, the above-described exemplary designs should not be considered to limit the scope of the appended claims.

What is claimed is:

1. A methanogen detection system, comprising:
a sample chamber configured to receive a capillary flow medium, the capillary flow medium configured to support a sample thereon, the sample comprising methanogens and each of the methanogens comprising Cofactor F₄₂₀;
a light source configured to produce a first light, the light source upstream to the sample chamber,
wherein the Cofactor F₄₂₀ of each of the methanogens absorbs the first light produced by the light source thereby resulting in excitation and relaxation of the Cofactor F₄₂₀ of each of the methanogens, and
wherein excitation and relaxation of the Cofactor F₄₂₀ of each of the methanogens generates a second emission light from the Cofactor F₄₂₀ of each of the methanogens;
an optical filter downstream from the second emission light, wherein the optical filter is configured to transmit a band of the second emission light;
a first light detector downstream from the second emission light and the optical filter, wherein the first light detector is configured to detect an intensity of the second emission light transmitted through the optical filter; and
a computing device in communication with the first light detector, wherein the computing device is configured to generate a concentration of the methanogens in the sample based on the intensity of the second emission light detected by the first light detector.

2. The methanogen detection system of claim 1, further comprising:
the capillary flow medium within the sample chamber.

3. The methanogen detection system of claim 2, wherein the capillary flow medium comprises:
a sample loading portion;
a hydrophobic partition downstream from the sample loading portion; and
a control portion downstream from the hydrophobic partition.

4. The methanogen detection system of claim 3, further comprising:
a second light detector downstream from the optical filter, wherein the second light detector is configured to detect an intensity of a third light produced by the light source and transmitted through the optical filter from the control potion of the capillary flow medium.

5. The methanogen detection system of claim 4, further comprising:
a power supply operatively connected to each of the light source, the first and second light detector, and the computing device.

6. The methanogen detection system of claim 5, wherein the power supply is a rechargeable battery.

7. The methanogen detection system of claim 5, further comprising:
a housing enclosing each of the sample chamber, the light source, the optical filter, the first and second light detector, the computing device, and the power supply.

8. The methanogen detection system of claim 7, wherein the housing defines an aperture extending through the housing, the aperture comprising a window configured to provide a view of the sample chamber.

9. The methanogen detection system of claim 7, wherein the housing defines a capillary flow medium slot, the slot configured to receive the capillary flow medium.

10. The methanogen detection system of claim 3, wherein the sample loading portion is configured to receive the sample,
wherein the sample is configured to flow on the sample loading portion toward the hydrophobic partition, and
wherein the hydrophobic partition is configured to restrain the sample from flowing to the control portion.

11. The methanogen detection system of claim 1, wherein the first light produced by the light source comprises a wavelength range of 420±2.5 nm.

12. The methanogen detection system of claim 1, wherein the optical filter is configured to transmit a band of the second emission light comprising a wavelength range of 470±2.5 nm.

13. The methanogen detection system of claim 1, wherein the light source comprises a plurality of light-emitting diodes (LEDs).

14. The methanogen detection system of claim 4, wherein each of the first and second light detector comprises a photodiode.

15. The methanogen detection system of claim 2, wherein the sample further comprises a lysis solution configured to break down a cell wall of each of the methanogens.

16. The methanogen detection system of claim 7, further comprising:
a port extending through the housing and operatively connected to each of the light source, the first and second light detector, and the computing device,
wherein the port is configured to receive and transfer power and receive, transfer, and output data.

17. The methanogen detection system of claim 3, wherein the capillary flow medium further comprises:
a user handling portion separate from the sample loading portion.

18. The methanogen detection system of claim 3, wherein the control portion is loaded with at least one fluorophore or at least one fluorochrome.

19. The methanogen detection system of claim 2, wherein the capillary flow medium is at least one of the following: chromatography paper, a lateral flow device, a lateral flow strip, or any combination thereof.

20. The methanogen detection system of claim 7, wherein the housing comprises:
a first chamber enclosing each of the capillary flow medium, the sample chamber, the light source, the optical filter, and the first and second light detector; and
a second chamber enclosing each of the computing device and the power supply.

21. A method of detecting methanogen using the methanogen detection system of claim 1, comprising:
providing a sample onto a capillary flow medium, the sample comprising one or more methanogens and each of the methanogens comprising Cofactor F₄₂₀;
providing the capillary flow medium into the sample chamber of the methanogen detection system,
irradiating the sample with the first light of the methanogen detection system;
detecting an intensity of a second emission light emitted from the Cofactor F₄₂₀ of each of the methanogens; and
generating a concentration of the methanogens in the sample based on the intensity of the second emission light.
